(19) United States
(12) Patent Application Publication
(10) Pub. No.: US 2012/0203236 A1
(43) Pub. Date: Aug. 9, 2012

(54) SURGICAL GUIDE

(76) Inventor: Alexander C. Mamourian, Wayne, PA (US)

(21) Appl. No.: 13/366,042

(22) Filed: Feb. 3, 2012

Related U.S. Application Data
(60) Provisional application No. 61/439,655, filed on Feb. 4, 2011.

(51) Int. Cl. A61B 17/17; A61B 17/00

(52) U.S. Cl. 606/96; 606/1

(57) ABSTRACT

A surgical guide and methods for facilitating the insertion of at least a portion of a surgical instrument into an intracranial ventricle and/or drilling a burr hole to access an intracranial ventricle are provided. The surgical guide includes a frame member adapted to be positioned over a portion of a patient's head, two plugs adapted to be positioned within a patient's external auditory meatuses and a guide member attached to a portion of the frame member for receiving the surgical instrument and ensuring the proper trajectory for inserting the surgical instrument at an appropriate angle in the plane of the external auditory meatuses.

This application is a non-provisional of and claims benefit of priority to U.S. Provisional Application No. 61/439,655, filed Feb. 4, 2011, the entire disclosure of which is hereby incorporated by reference as if set forth fully herein.

### BACKGROUND OF THE INVENTION

1. Field of the Invention

The invention is directed to a surgical guide and a method for facilitating the placement of a surgical instrument in an intracranial ventricle and/or the placement of a burr hole for accessing the intracranial ventricle. In particular, the invention is adapted for facilitating bedside placement of an external ventricular drain, placement of a ventricular catheter of a ventriculoperitoneal shunt and placement of a chemotherapy catheter with a reservoir.

2. Description of the Related Technology

Insertion of an external ventricular drain is among the most common techniques employed in neurosurgery with over 25,000 procedures being performed in the U.S. annually. The procedure involves drilling a single burr hole into the patient's cranium, identifying an optimal trajectory for inserting a ventricular drain and inserting a catheter to measure pressures and drain cerebrospinal fluid, wherein catheter placement typically involves referencing the position of external facial features or features of the skull. Accurately determining the location of the lateral ventricle and inserting a catheter at the appropriate trajectory and to an appropriate depth are critical to the proper placement of an external ventricular drain. Current techniques for identifying the location of the ventricle and an appropriate insertion trajectory, however, are imprecise and often result in multiple drain placement attempts with the accompanying potential for causing trauma and hemorrhage or placement of the drain in suboptimal locations that increases the likelihood of short term replacement.

Furthermore, in circumstances requiring free hand emergency placement of a ventricular drain, a surgeon must rely on external anatomical reference points, such as the medical canthus, external auditory canal, nasion and coronal suture, to provide guidance in identifying the location of the lateral ventricle. The surgeon then endeavors, via a freehand approach, to insert the catheter, using visual guidance for selecting an insertion trajectory. Because this method is inexact and accurate placement is dependent upon surgeon experience, while recognizing that the procedure is often performed by the least experienced members of the neurosurgical team, significant complications occur in up to 5% of such procedures.

Retrospective studies of external ventricular drain placement indicate that placement at the bedside is less than optimal and that suboptimal placement and multiple passes are commonplace. As reported in Huyette, D. R., et al., “Accuracy of the freehand pass technique for ventriculostomy catheter placement: retrospective assessment using computed tomography scans,” J. Neurosurg. 108:88-91, 2008, on average two passes were necessary for each successful placement, and 22% of the time the catheter was not in the ventricle. Inherent in averaging, some patients may require five or more attempts. Toma, A. K., et al., “External ventricular drain insertion accuracy: Is there a need for change in practice?,” Neurosurgery 65:1187-1201, 2009, disclosed the results of a 183 patient study examining external ventricular drain placement using a freehand approach. The catheter tip was found to be located in a cerebrospinal fluid space other than the frontal horn in half the patients, and in another 10% of the patients, the tip was in the brain parenchyma. In those patients with optimal catheter tip location in the frontal horn, revision was necessary in 25% of the patients compared with a revision rate of 44% in the 75% of patients with a suboptimal catheter position.

Although the use of stereotactic techniques in conjunction with a pre-operative computed tomography (CT) scan of a patient's head, such as that described in U.S. Pat. No. 6,132,437, more accurately identifies the location of intracranial targets in comparison to a freehand approach, a stereotactic approach adds time and complexity to the procedure. Because a surgeon must orient such stereotactic frames based on coordinates obtained from the CT scan, errors introduced by motion on CT images, the potential for human error in urgent situations and inexperienced operators working late at night are factors that may compromise the operator's ability to achieve the promised accuracy at the time the drain is placed.

Frameless stereotaxy approaches to facilitate ventricular insertion are also available. These methods, however, require expensive and relatively stationary equipment that usually necessitates transportation of the patient to where the equipment resides. Frameless stereotaxy techniques, therefore, are limited to use in critical care facilities and operating rooms and are not commonly available for bedside use, particularly at night, or for emergency situations.

Freehand guides, such as that described in U.S. Pat. No. 4,613,324, have also been used to provide some directional assistance with freehand ventricular catheter insertion. When using such freehand guides, the catheter is directed along a trajectory perpendicular to the angulation of the skull using a burr hole drilled at the same angulation. This device, however, does not allow accommodation for ventricular shifts or irregularities in the skull. Therefore it may not provide an optimal insertion trajectory since it assumes a normal skull and has not the capability to adjust the trajectory of the drain based on imaging.

In view of these deficiencies, there exists the need for an improved guide that rapidly and simply achieves a more accurate trajectory for the placement of a catheter in an intracranial ventricle, thereby reducing the time required for external ventricular drain procedures and reducing the likelihood of complications as well as the risk of infection from catheter exchanges due to drain misplacement. Additionally, there is a need to develop a device which allows image-guided external ventricular drain insertion to be conducted at a patient's bedside or in emergency situations, thereby improving placement precision and reducing the risk of adverse consequences for patients.

### SUMMARY OF THE INVENTION

In a first aspect, the present invention is directed to a surgical guide. The surgical guide includes a first frame member having a substantially U-shaped configuration adapted to be positioned over a portion of an individual's head, two plugs adapted to be positioned within an individual's external auditory meatuses and a guide member positioned along a portion of the first frame member. The guide member includes a passage for receiving a surgical instrument, wherein the first frame member and the guide member cooperate to ensure that a longitudinal axis of a distal portion of the passage is oriented in a plug plane that passes through a line intersecting a portion of each the two plugs that is positioned within the external auditory meatus of an individual to allow for insertion of the surgical instrument at a desired trajectory based on a fixed angle from the midline.

In a second aspect, the invention is directed to a method for positioning a portion of a surgical instrument in an intracranial ventricle. The method involves the steps of positioning two plugs of a surgical guide within an individual's external auditory meatuses. The surgical guide includes a first frame member having a substantially U-shaped configuration and a guide member including a passage for receiving the surgical instrument. A longitudinal axis of a distal portion of the passage in the guide member is oriented in a plane that passes through a longitudinal axis of an individual's external auditory meatuses allowing for insertion of the surgical instrument at a desired trajectory based on a fixed angle from the midline. The method further involves aligning the guide member with a burr hole drilled into the individual's cranium, inserting the surgical instrument through the passage in the guide member and into the burr hole and positioning a portion of the surgical instrument within the intracranial ventricle.

In a third aspect, the invention is directed to a method for accessing an intracranial ventricle. The method involves the steps of positioning two plugs of a surgical guide within an individual's external auditory meatuses. The surgical guide includes a first frame member having a substantially U-shaped configuration and a guide member including a passage for receiving the surgical instrument. A longitudinal axis of a distal portion of the passage in the guide member is oriented in a plane that passes through a longitudinal axis of an individual's external auditory meatuses to allow for insertion of the surgical instrument at a desired trajectory based on a fixed angle from the midline. The method further involves identifying a location of the intracranial ventricle, identifying a burr hole drill site based on the identified location of the intracranial ventricle, aligning the guide member with the burr hole drill site, inserting a drill bit into the passage and drilling a burr hole in an individual's cranium.

### BRIEF DESCRIPTION OF THE DRAWINGS

FIG. 1 shows a perspective view of a first embodiment of a surgical guide including a first frame member, two external auditory meatus plugs and a guide member.

FIG. 2 shows a perspective view of the surgical guide of FIG. 1 further including extension members and stops attached to the first frame member.

FIG. 3 shows a perspective view of a second embodiment of a surgical guide including a first frame member, a second frame member pivotally adjustable with respect to the first frame member, two external auditory meatus plugs and a guide member.

FIG. 4 shows a perspective view of a third embodiment of a surgical guide including a first frame member, a second frame member pivotally adjustable through a limited range of angles with respect to the first frame member, two external auditory meatus plugs and a guide member.

FIG. 5 shows a perspective view of a fourth embodiment of a surgical guide including a first frame member, a second frame member pivotally fixed with respect to the first frame member, two external auditory meatus plugs and a guide member.

### DETAILED DESCRIPTION OF THE PREFERRED EMBODIMENT(S)

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

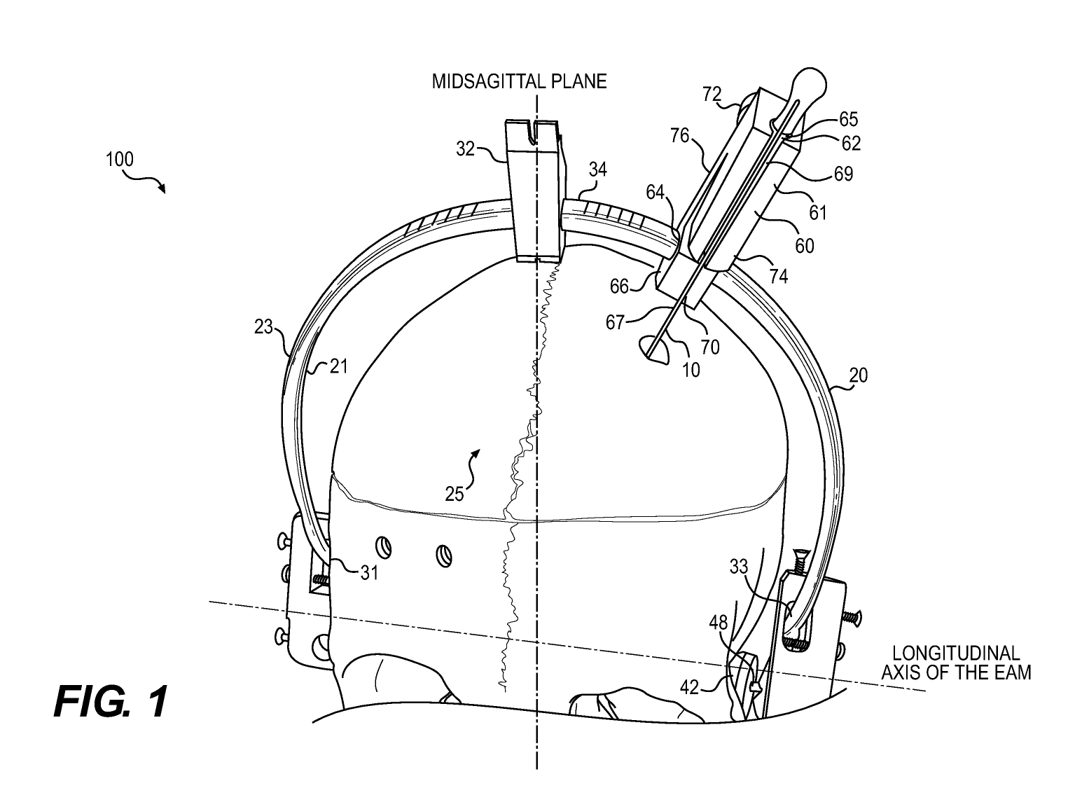

For purposes of the present invention, the term, “external auditory meatuses plane” or “EAM plane,” as used herein, refers to the coronal plane passing through a longitudinal axis of both of an individual's external auditory meatuses. When the surgical guide of the present invention is not positioned on an individual for use, reference is made to any plane in space which passes through a line intersecting a portion of each of the two plugs adapted for being positioned within the external auditory meatuses of an individual. As a result, when the surgical guide of the present invention is mounted to an individual's head, the line intersecting a portion of each the two plugs that is positioned within the external auditory meatuses of an individual is the same as the longitudinal axis of an individual's external auditory meatuses, as shown in FIG. 1.

As used herein, “plug plane” refers to any plane in space passing through the line intersecting a portion of each of the two plugs that is positioned within the external auditory meatuses of an individual. Preferably, the plug plate passes through the line intersecting corresponding portions of, more preferably, corresponding central portions of each of the two plugs positioned within the external auditory meatuses of an individual. Therefore, when the device of the invention is positioned on an individual with the plugs positioned within the external auditory meatuses of an individual, the plug planes and the EAM planes are the same.

As used herein, “surgical instrument” refers to any surgical device. Preferably, surgical instrument refers to a surgical device adapted to be introduced into the brain of an individual or a surgical device that enables and/or assists an apparatus to be introduced into the brain of an individual. Exemplary surgical instruments may include, but are not limited to, a catheter, a needle, a shunt, a drill, a cannula, a trocar, a probe, an electrode, a deep brain stimulator, a heating element, a sensor, an ablator and a biopsy device.

The present invention is directed to novel surgical guides and methods for facilitating the insertion of at least a portion of a surgical instrument 10 into an intracranial ventricle and/or drilling a burr hole to access an intracranial ventricle. Referring now to the drawings, wherein like reference numerals designate corresponding structures throughout the drawings, and referring in particular to the exemplary embodiment shown in FIG. 1, surgical guide 100 includes a first frame member 20 having a substantially U-shaped configuration adapted to be positioned over a portion of a patient's head, two plugs 40, 42 adapted to be positioned within a patient's external auditory meatuses and a guide member 60 is slidably mounted on a portion of first frame member 20. Guide member 60 includes a passage 62 for receiving a surgical instrument 10 and for orienting the trajectory of insertion of surgical instrument 10 into the patient. The longitudinal axis of at least a distal section 70 of passage 62 is oriented in a plug plane by virtue of a combination of the structure of frame member 20 and guide member 60, and thereby is also oriented in an EAM plane. Surgical guide 100 uses the location of a pre-drilled burr hole, the longitudinal axis of the external auditory meatuses which is coincident with a line intersecting a portion of each the two plugs 40, 42 that is positioned within the external auditory meatuses and the midsagittal plane of the body to accurately locate an intracranial ventricle and relies on the EAM plane to appropriately orient the trajectory of surgical instrument 10. Preferably, the burr hole is pre-drilled in the usual fashion at Kocher's point. The structure of surgical guide 100 and orientation of guide member passage 62 therefore may be used to reliably identify the location of an intracranial ventricle, specifically the foramen of monro of the lateral ventricle, and achieve an optimal surgical instrument insertion trajectory. By virtue of its simple structure, and by orienting the longitudinal axis of passage 62 within an EAM plane, surgical guide 100 is adapted for the accurate, effective and quick bedside placement of a portion of surgical instrument 10 into an intracranial ventricle without requiring CT scans of the patient or additional instrumentation.

In an exemplary embodiment, surgical guide 100 limits the posterior to anterior adjustment of first frame member 20 and/or the medial to lateral adjustment of guide member 60 in order to simplify use, thereby enabling the rapid placement of surgical instrument 10 and minimizing the possibility of human error in identifying the proper location and trajectory of insertion. The surgical guide 100 of the present invention may be particularly well suited to facilitate the bedside placement of an external ventricular drain or the ventricular catheter of a ventriculoperitoneal shunt.

In the embodiment shown in FIG. 1, first frame member 20 forms a substantially U-shaped configuration adapted to be coupled to a patient's head. Preferably, first frame member 20 may have a substantially arcuate or arch shaped configuration. First frame member 20 may be constructed from any suitable materials, such as plastics, metals or ceramics, and may have any suitable dimensions for forming a structure that rigidly connects plugs 40, 42 and rigidly supports guide member 60. In an exemplary embodiment, first frame member 20 may be configured as a bar, with a rectangular or cylindrical cross-section, that is bent and/or formed to have a substantially U-shaped configuration.

End portions of first frame member 20 may have any suitable configuration adapted to be positioned proximate to the side of a patient's head. In one embodiment, end portions of first frame member 20 may have a substantially linear structure and are oriented substantially opposite to and parallel with respect to one another. Alternatively, end portions of first frame member 20 may have a curved structure such that the end portions have inwardly facing and opposing concave curvatures that at least partially conform to the rounded sides of a patient's head, as shown, for example, in FIG. 1.

A portion of first frame member 20 adapted to be positioned over the crown of a patient's head connects the end portions of first frame member 20. Scaled markings 34 denoting distance, measured in units such as inches and/or centimeters, may be positioned along a length of at least the central region of first frame member 20.

In one embodiment, first frame member 20 may optionally include a fixed or adjustable indicator 32 for identifying the midline of first frame member 20 or the midsagittal plane of a patient. Indicator 32 is preferably slidably positioned along a length of first frame member 20, and any standard locking mechanism 35, such as a clamp, clasp, latch, screw or combinations thereof, may be used to adjustably secure indicator 32 in place relative to first frame member 20.

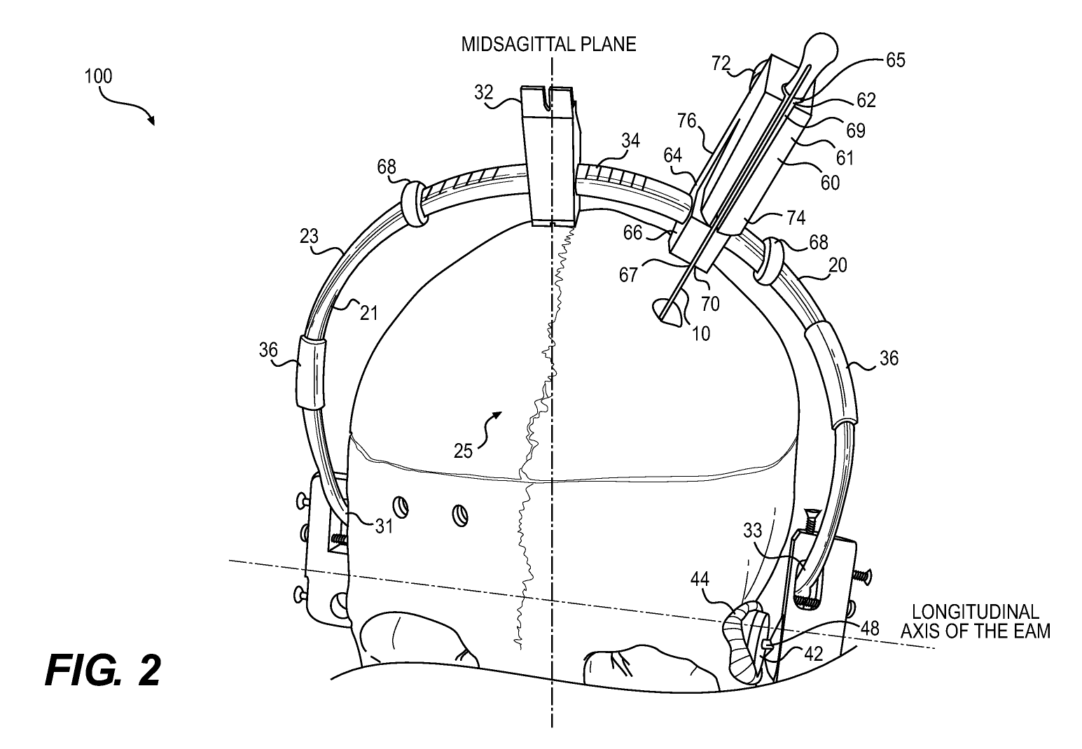

Optionally, first frame member 20 may also include a means for adjusting its length so as to fit patients of various head heights, as shown in FIG. 2. In an exemplary embodiment, first frame member 20 may include extension members 36 for adjusting the position and/or extending the length of first frame member 20. Extension members 36 may have any suitable configuration, such as guide rails that are slidably coupled to slidable end portions of first frame member 20 or sliding bars slidably positioned within the slots of guide rail configured end portions of first frame member 20. Extension members 36 may be positioned anywhere along the length of first frame member 20. In the embodiment shown in FIG. 2, width adjustment means may be configured as two extension members 36 located at opposing end portions of first frame member 20.

Similarly, first frame member 20 may optionally include a means for adjusting its width, not shown, to accommodate heads of various widths. In an exemplary embodiment, the width adjustment means is configured as an extension member 36 having the same structural features, function and means for coupling to a portion of first frame member 20 as discussed above may be coupled to first frame member 20. Preferably the width adjustment means is located on a portion of first frame member 20 adapted to be positioned over the crown of a patient's head.

As shown in FIG. 1, surgical guide 100 further includes a first plug 40 and a second plug 42 rigidly connected to first frame member 20 and adapted to be positioned within a patient's external auditory meatuses. First and second plugs 40, 42 may have any suitable shape and configuration adapted to be securely fitted within a patient's external auditory meatuses. In an exemplary embodiment, first and second plugs 40, 42 may have a generally spherical shape, oblong shape, rectangular shape, L shape or shape conforming to the spaced within an external auditory canal meatuses. Preferably, the edges and distal tip of first and second plugs 40, 42 are rounded. First and second plugs 40, 42 may be constructed from any suitable rigid material, such as plastics, metals and ceramics, adapted for comfortably securing plugs 40, 42 within the external auditory meatuses and rigidly positioning first and second plugs 40, 42 relative to first frame member 20.

Optionally, foam and/or fabric coverings 44 may be placed over first and second plugs 40, 42 to enhance patient comfort and further ensure that plugs 40, 42 are secured within the external auditory canal. Covering 44 may be integrally formed with and affixed to first and second plugs 40, 42. Alternatively, a disposable covering 44 may be removably attached to an exterior surface of first and second plugs 40, 42, providing an additional sanitation barrier.

First and second plugs 40, 42 may be directly or indirectly connected to an interior surface 21 or exterior surface 23 of first frame member 20 at a location proximate a mid to lower region of first frame member 20. In one embodiment, first and second plugs 40, 42 are directly connected to an interior surface 21 proximate to first and second ends 31, 33, protruding towards an interior space 25 defined by first frame member 20. In another embodiment, first and second plugs 40, 42 are positioned on rigid first and second posts 46, 48 extending from an interior surface 21 of first frame member 20, preferably proximate to first and second ends 31, 33. First frame member 20, first and second posts 46, 48 and plugs 40, 42 may be rigidly connected, forming an integral system.

When plugs 40, 42 are positioned within a patient's external auditory meatuses, a line intersecting a portion of each the two plugs 40, 42 that is positioned within the external auditory meatus corresponds to the longitudinal axis of the patient's external auditory meatuses. This provides a reference for accurately locating the intracranial ventricle and for determining the appropriate trajectory at which surgical instrument 10 is to be inserted.

As shown in FIG. 1, surgical guide 100 further includes a guide member 60 having any suitable dimension and configuration enabling it to be coupled to first frame member 20 and adapted for receiving and orienting a trajectory of surgical instrument 10, such as a drain. Guide member 60 is preferably adjustably coupled to first frame member 20 so that guide member 60 can be slidably positioned along first frame member 20. Furthermore, guide member 60 may be coupled to first frame member 20 such that it is positioned along a side of or through an opening defined in the body of first frame member 20.

In one embodiment, a slot 64 may be defined in and pass through an exterior surface 61 of guide member 60 that is sized and configured to be permanently or removably coupled to an edge of first frame member 20. Slot 64 may have any configuration that mates with and corresponds to an edge of first frame member 20, such as a U-shaped slot or rail guide. When slot 64 is coupled to the edge of first frame member 20, guide member 60 is slidably positioned along a side of first frame member 20.

In another embodiment, guide member 60 includes a protrusion adapted to be inserted within and slidably coupled to an elongated slot inset with respect to an edge, interior surface 21 or an exterior surface 23 of first frame member 20. The protrusion and elongated slot may have any suitable corresponding mating configuration, such as a protruding T-shaped rail and corresponding rail guide. The slot and protrusion may be coupled in a manner such that guide member 60 is slidably positioned along a side of first frame member 20.

In yet another embodiment, first frame member 20 may include an elongated hole that extends along a portion of or the entirety of its length. In this embodiment, at least two corresponding slots positioned on opposing sides of guide member 60 may be slidably coupled to the inner edges of first frame member 20 that form the elongated hole, such that guide member 60 is inserted within and slidably positioned along a central region of first frame member 20.

Guide member 60, specifically via a slot 64 of guide member 60, may be removably attached to first frame member 20. Two or more guide members 60, each adapted to receive different types of surgical instruments 10 may then be removably and interchangeably coupled to first frame member 20. Alternatively, guide member 60, specifically passage 62, may be configured to receive or may be adjusted to receive a wide variety of different surgical instruments 10. A first securing member 66, such as a clamp or screw located proximate to slot 64 may be used to releasably secure guide member 60 to a location along first frame member 20. In order to securely retain guide member 60 to first frame member 20, first securing member 66 may, for example, narrow the opening of slot 64 or press a portion of first frame member 20 against an interior surface of guide member 60 that defines slot 64.

Optionally, two stops 68 having a structure and dimension suitable for preventing the slidable adjustment of guide member 60 may be formed along and protrude out from an interior surface 21 and/or exterior surface 23 of first frame member 20, thereby restricting the location over which guide member 60 may be adjustably positioned. In an exemplary embodiment shown in FIG. 2, stops 68 are arranged to restrict guide member 60 to being adjustably positioned over a region suitable for locating an intracranial ventricle. In one embodiment, stops 68 are located about 4 cm to about 10 cm to the left and to the right of the midline of first frame member 20. Preferably, stop 68 is positioned about 3.5 cm to about 7 cm to the right of the midline of first frame member 20, since this is the usual approach side and as the burr hole is typically placed about 3 cm from the midline.

A passage 62 extending through a portion of guide member 60 is adapted for receiving and guiding the trajectory of surgical instrument 10. As shown in FIG. 1, the longitudinal axis of passage 62 is oriented by first frame member 20 such that it is situated within a plug plane, and thereby also oriented in an EAM plane. Specifically, the longitudinal axis of at least a distal section 70 of passage 62, proximate to interior space 25 and oriented in a direction pointing towards a patient's cranium when surgical guide 100 is mounted on a patient, is oriented within a plug plane. This feature of the invention establishes that the trajectory of surgical instrument 10 received within guide member 60 will always be oriented in the EAM plane when the device 100 is placed on a patient's head with plugs 40, 42 located in the external auditory meatuses, thereby increasing the likelihood of accurately placing surgical instrument 10 within the intracranial ventricle. Slot 64 may be angled with respect to first frame member 20 and the body of guide member 60 in any manner necessary to orient the longitudinal axis of passage 62 or at least a distal section 70 of passage 62 within a plug plane. Additionally, the angular orientation of guide member 60 relative to first frame member 20 and plugs 40, 42 is fixed to ensure that the longitudinal axis of passage 62 is fixedly oriented within an EAM plane when the device 100 is placed on a patient's head with plugs 40, 42 located in the external auditory meatuses.

In one embodiment, passage 62 may be defined in and positioned through the body of guide member 60 so as to form a partially enclosed channel with a proximal opening 65 for inserting surgical instrument 10 and a distal opening 67 through which surgical instrument 10 is guided. Alternatively, passage 62 may have a substantially open configuration, wherein a length of the passage 62 is partially formed in and penetrates an exterior surface of guide member 60, forming a side opening 69. In this embodiment, passage 62 is open to and communicates with an open space surrounding exterior surface 61. This side opening 69 may allow surgical instrument 10 to be removed from guide member 60 through a side surface of guide member 60 and remain within the EAM plane. In one embodiment, a flexible or movable gate, such as a sliding or pivotally articulating gate, may be positioned over side opening 69 to allow easy separation of guide member 60 and surgical guide 100 from surgical instrument 10, such a catheter, after placement. Guide member 60 may further include a clamp 72 for releasably securing the surgical instrument within the longitudinal passage 62. In this embodiment, the space defined by passage 62 is open to an area surrounding an exterior surface 70 of guide member 60 allowing the ingress and egress of a surgical instrument through the longitudinal side of guide member 60. Guide member 60 may further include a second securing member 72, such as a clamp or screw, proximate to passage 62 for releasably securing surgical instrument 10 within the passage 62. Second securing member 72 may function to releasably press against a portion of or constrict passage 62 to secure surgical instrument 10.

In one embodiment shown in FIG. 1, guide member 60 has a rectangular body that is oriented substantially perpendicular to and coupled to an interior and/or exterior surface 21, 23 of first frame member 20. Slot 64, defined in a side of guide member 60, is aligned with and encloses an edge of first frame member 20. A substantially cylindrical passage 62 extends through the length of guide member 60, perpendicular to slot 64. Passage 62 is partially formed in an exterior surface 61 of guide member 60 such that the length of passage 62 is open to a space surrounding exterior surface 61 and forms side opening 69. A second securing member 72, configured as a clamp surrounding a portion of passage 62, functions to releasably retain surgical instrument 10 within passage 62. In this embodiment, the longitudinal axis of passage 62 is substantially perpendicular to an interior and/or exterior surface 21, 23 of first frame member 20 and is fixed such that at least a distal portion of passage 62 is always oriented within a plug plane.

In a second embodiment, guide member 60 may have a two part structure including a first guide portion 74 for receiving surgical instrument 10 and an adjoining second connector portion 76 coupled to first frame member 20. In an exemplary embodiment, the body of guide member 60 may have a substantially L- or T-shaped configuration. In an exemplary embodiment, the second connector portion 76 of an L-shaped guide member 60 may be clamped to first frame member 20 and oriented substantially parallel to the skull while first guide portion 76 is angled towards the skull.

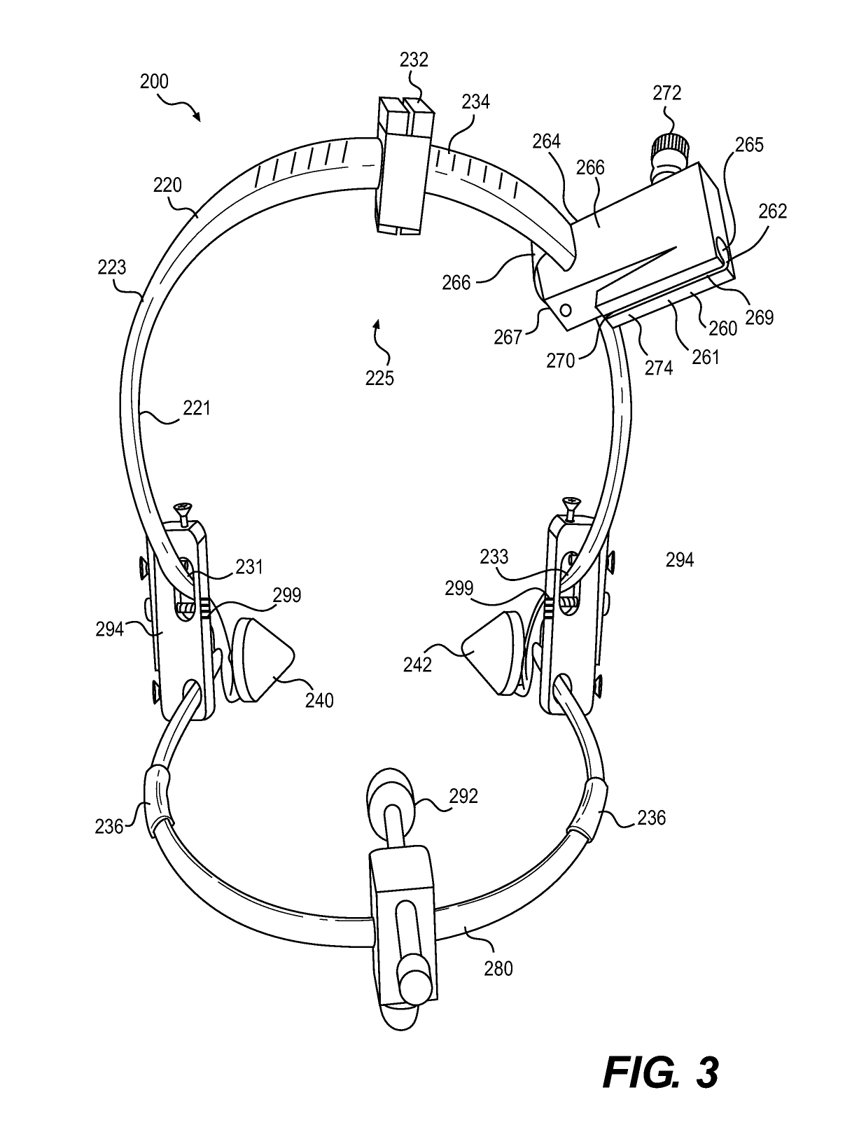

In a second embodiment of the invention shown in FIG. 3, a surgical guide 200 may be designed to facilitate the placement of a surgical instrument 210 into an intracranial ventricle and/or facilitate drilling a burr hole to access an intracranial ventricle. Surgical guide 200 may include similar components as surgical guide 100, including a substantially U-shaped first frame member 220 adapted to be positioned over a portion of a patient's head, two plugs 240, 242 adapted to be positioned within a patient's external auditory meatuses and a guide member 260 that is slidably positioned along a portion of first frame member 220 for orienting the trajectory of surgical instrument 210. Preferably, first frame member 220 and guide member 260 are the same as and have the same components as first frame member 20 and guide member 60. In this embodiment, surgical guide 200 may further include a second frame member 280 having a substantially U-shaped configuration adapted to be positioned against a patient's nasion and an adjustment mechanism 294 for adjusting the angle between first and second frame members 220, 280. In addition to providing additional structural support for surgical guide 200 when mounted on a patient's head, second frame member 280 also functions as a reference point for facilitating the process of positioning first frame member 220 to locate an intracranial ventricle and locating an appropriate burr hole drill site to access the intracranial ventricle.

As shown in FIG. 3, second frame member 280 may have a substantially arcuate or arch shaped configuration. Second frame member 280 may have any suitable configuration adapted for end portions of second frame member 280 to be horizontally positioned adjacent to the side of a patient's head. In one embodiment, end portions of second frame member 280 may have a substantially linear structure and may be oriented substantially opposite to and parallel with respect to one another. Alternatively, end portions of second frame member 280 may have a curved structure such that the end portions have inwardly facing and opposing concave curvatures that at least partially conform to the rounded sides of a patient's head. Second frame member 280 is adapted to be positioned across the bridge of a patient's nose and against the patient's nasion. Second frame member 280 may include a nasion rest 292 that is connected to and protrudes out from an interior surface thereof. In an exemplary embodiment, nasion rest 292 may be configured as post, preferably with a concave distal end having a surface that conforms to and supports the nasion.

Optionally, second frame member 280 may include a means for adjusting its length and/or width so as to adjustably fit a variety of patients. In an exemplary embodiment, extension members 236 having the same structural features, function and coupling means as extension member 36, discussed above, may be positioned along any portion of second frame member 280 for adjusting a position and/or extend a length thereof.

Coupled to first and/or second frame members 220, 280 is an adjustment mechanism 294 that enables the precise angular adjustment between first and second frame members 220, 280. Adjustment mechanism 294 may be any convention device suitable for adjustably positioning, locking and unlocking the position of first frame member 220 relative to second frame member 280. In an exemplary embodiment, adjustment means 294 may be a ratchet comprising a plurality of fasteners 296, such as teeth, hooks or latches that may be removably attached to a corresponding mating structure 298, such as a notch or aperture, positioned along an edge of first or second frame members 220, 280. In one embodiment, a pair of such ratchets may be located on the opposite sides of first and second frame members 220, 280. Scaled markings 299 denoting the angle formed between first and second frame members 220, 280 may be positioned along the body of adjustment mechanism 294, for example, along an exterior face of a ratchet member allowing a surgeon to precisely adjust the angle between first and second frame members 220, 280.

In this embodiment, first and second frame members 220, 280 may be pivotally adjusted relative to one another over a range of about 0° to about 360°, preferably over a range of about 0° to about 270°, more preferably, over a range of about 0° to about 245° and most preferably, over a range of about 0° to about 180°. This embodiment of surgical guide 200 may be particularly well suited to locate intracranial ventricles that have been distorted by intracranial masses, such as a hematoma. In such cases, location of the shifted intracranial ventricles may be obtained by imaging a patient's head. For example, a helical CT scan of a patient's head and a 3D image processing system may be used to locate and calculate the precise coordinates of the ventricle. A surgical guide 200 of the present invention may then be used to map and accurately guide surgical instrument 210 to the ventricle. The use of a CT scan in conjunction with the pre-calibrated angular degree of freedom of surgical guide 200 may therefore further improve the chances of accurate first-time drain placement.

When surgical guide 200 of the present invention is used as a drill guide or as both a drill guide and a surgical instrument placement guide, the burr hole drilling location can be determined based on the CT scan data and by mapping the appropriate drill location using a reference plane based on anatomical features of the patient's skull. Surgical guide 200 operates as a coordinate system for locating the optimal drill site based on the CT scan data.

Furthermore, when surgical guide 200 is used as a drill guide, guide member 260 may be adapted to receive different types of surgical instruments 210, including drills, catheters and needles. In another embodiment, two or more guide member 260, each adapted to receive a different surgical instrument 210, such as a drill or catheter, may be removably and interchangeably attached to first frame member 220.

Surgical guide 200 uses the nasion against which second frame member 280 is positioned, the longitudinal axis of the external auditory meatuses corresponding to the line between portions of plugs 240, 242 that are inserted in the external auditory meatuses of the patient and the midsagittal plane, as identified by indicator 232 to accurately locate an intracranial ventricle and drill a burr hole to access the intracranial ventricle. Without wishing to be bound by theory, surgical guide 200 is able to accurately locate an intracranial ventricle and appropriate drill site as well as establish the proper surgical instrument insertion trajectory by virtue of the fact that the location of the frontal horn with respect to the nasion, the longitudinal axis of the external auditory meatuses and the midsagittal plane are substantially uniform in most patients. The structure of surgical guide 200, reference guides of surgical guide 200 and orientation of guide member passage 262 within a plug plane, and hence an EAM plane when placed on a patient, may therefore be used to reliably identify the location of an intracranial ventricle, identify an appropriate drill site to access an intracranial ventricle and achieve an appropriate surgical instrument insertion trajectory to reach the intracranial ventricle.

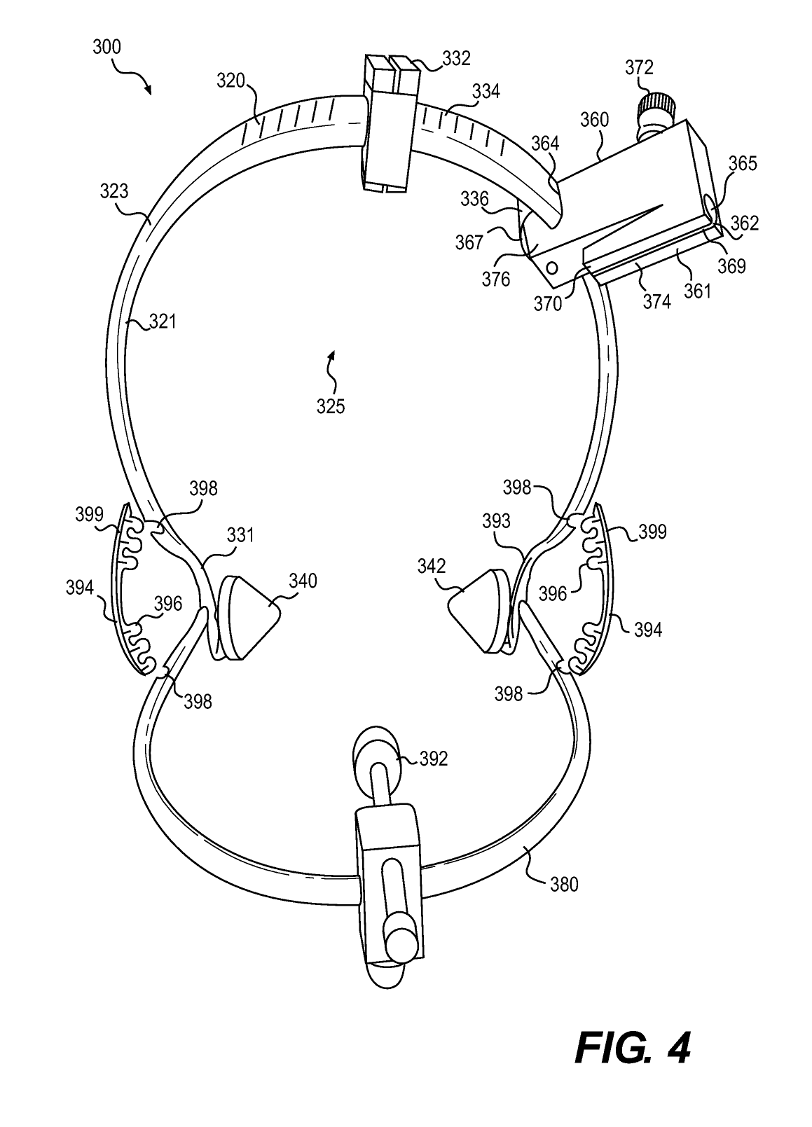

In a third embodiment shown in FIG. 4, a surgical guide 300 may be designed to facilitate the placement of a surgical instrument 310 into an intracranial ventricle and/or facilitate drilling a burr hole to access an intracranial ventricle. Surgical guide 300 may include the same or substantially similar components to surgical guide 200, including a first U-shaped frame member 320 adapted to be positioned over a portion of a patient's head, a second U-shaped frame member 380 positioned against a patient's nasion, an adjustment mechanism 394 for adjusting the angle between first and second frame members 320, 380, two plugs 340, 342 adapted to be positioned within a patient's external auditory meatuses and a guide member 360 that is slidably positioned along a portion of first frame member 320 for orienting the trajectory of surgical instrument 310. In this embodiment, the adjustment mechanism 394 is restricted such that first and second frame members 320, 380 may only be oriented in a limited number of positions to more quickly deploy, enable easier use and limit the potential for human error when using surgical guide 300. Scaled markings 399 on adjustment mechanism 394 may indicate the angle between the first and second frame members 320, 380.

The range of angles over which first and second frame members 320, 380 may be positioned may be limited to a range of select angles that facilitate the location of an intracranial ventricle. In one embodiment, to facilitate the location of an intracranial ventricle, specifically the foramen of monro of the lateral ventricle, for the placement of an external ventricular drain, first and second frame members 320, 380 may be pivotally positioned relative to one another to form an angle of about 55° to about 65°. In another embodiment, the angle between first and second frame members 320, 380 is about 40° to about 65°, preferably, about 40° to about 55°, in order to facilitate the location of a ventricle for the placement of a ventricular drain. Preferably, first and second frame members 320, 380 may be pivotally positioned relative to one another over the aforementioned angular ranges in any desired increments, including increments of about 1° to about 10°, more preferably, about 1° to about 5°.

As shown in the exemplary embodiment of FIG. 4, this limited angular adjustment of first and second frame members 320, 380 may be accomplished by restricting the location of fasteners 396 on a ratchet configured adjustment mechanism 394. Fasteners 396 may be positioned only along a middle region of the ratchet limiting the maximum angle to which first and second frame members 320, 380 may be set. Furthermore, fasteners 396 may be arranged in two groups spaced apart from one another to limit the minimum angle to which first and second frame members 320, 380 may be set.

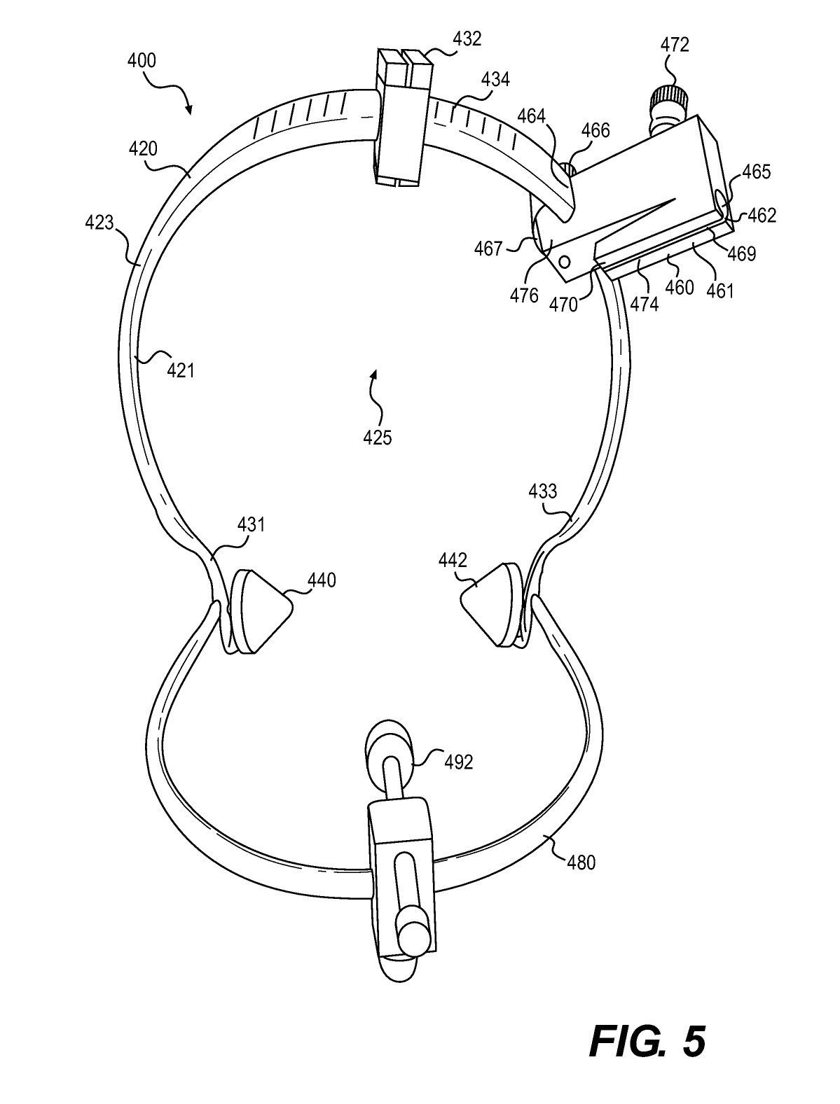

In a fourth embodiment shown in FIG. 5, a surgical guide 400 may be designed to facilitate the placement of surgical instrument 410 into an intracranial ventricle and/or facilitate drilling a burr hole to access an intracranial ventricle. Surgical guide 400 may include the same or substantially similar components to surgical guide 200, including a first U-shaped frame member 420 adapted to be positioned over a portion of a patient's head, a second U-shaped frame member 480 positioned against a patient's nasion, two plugs 440, 442 adapted to be positioned within a patient's external auditory meatuses and a guide member 460 that is slidably positioned along a portion of first frame member 420 for orienting the trajectory of surgical instrument 410. In this embodiment, however, first and second frame members 420, 480 are fixed relative to one another and thus this embodiment does not include an adjustment mechanism

As shown in FIG. 5, second frame member 480 is integrally formed with and fixed relative to first frame member 420. Preferably, distal ends 481, 483 of second frame member 480 may be affixed with distal end 431, 433 of first frame member 420. The fixed angle formed between first and second frame members 420, 480 facilitates the location of an intracranial ventricle. In one embodiment, to facilitate the location of an intracranial ventricle, specifically the foramen of monro of the lateral ventricle, for the placement of an external ventricular drain, the angle between first and second frame members 420, 480 is about 55° to about 65°. In another embodiment, the angle between first and second frame members 420, 480 is about 40° to about 65°, preferably, about 40° to about 55° in order to facilitate the location of a ventricle for the placement of a ventricular drain.

Surgical guides 100, 200, 300 and 400 of the present invention offer a number of advantages in comparison to the currently available technology. By relying on and referencing anatomical features that are substantially uniformly situated relative to one another in all patients, namely the nasion, longitudinal axis of the external auditory meatuses, and the midsagittal plane, the present invention enables the rapid and accurate location of intracranial ventricles. Moreover, adjustable versions of the present invention, such as surgical guides 200, 300, may also be used in conjunction with CT imaging to map the exact location of an intracranial ventricle, which may be useful when a shift or mass effect is present. In these situations, the location of the intracranial ventricle may be identified by imaging. The invention also provides a high probability of placing a surgical instrument in an intracranial ventricle by virtue of orienting the longitudinal axis of passage 62, 262, 362, 462 specifically a longitudinal axis of a distal portion 70, 270, 370, 470 of passage 62, 262, 362, 462, in a plug plane, thereby achieving an optimal insertion trajectory.

The accuracy achieved by the invention is expected to improve the speed and safety of procedures, such as external ventricular drain placement or ventriculoperitoneal shunt placement. Furthermore, the simple structure of the present invention enables the surgical guide to be rapidly deployed and easily used. The pre-calibrated and limited adjustment features of the present invention, particularly that of surgical guides 100, 300 and 400, reduces the chance of human error in placing a surgical instrument in an intracranial ventricle. The device is therefore calibrated to provide the necessary degree of freedom without an extensive setup procedure. As the invention may be constructed as a portable, inexpensive, disposable, plastic preformed apparatus, it may suitable for wide spread use in bedside placement of surgical instruments, particularly in emergency situations. Moreover, in addition to a surgical instrument placement guide, the present invention, particularly surgical guides 200, 300 and 400, may be suitable for use as a drill guide.

The present invention is also directed to a novel method for placing a surgical instrument in an intracranial ventricle. In particular, the invention is directed to a method for facilitating the placement of an external ventricular drain or the ventricular catheter of a ventriculoperitoneal shunt. An exemplary method of the present invention involves mounting a sterile surgical guide 100, 200, 300 or 400 of the present invention on a patient's head such that sterilized plugs 40, 42, 240, 242, 340, 342, 440, 442 are positioned within a patient's external auditory meatuses and sterilized first frame member 20, 220, 320, 420 is positioned over the crown of the patient's head. The plugs 40, 42, 240, 242, 340, 342, 440, 442 may be securely positioned with the external auditory meatuses such that a line between portions of the plugs inserted in the external auditory meatuses of the patient aligns with the longitudinal axis of the external auditory meatuses. With respect to surgical guides 200, 300 and 400, second frame member 280, 380, 480 may be positioned against a patient's nasion to provide further structural support when surgical guide 200, 300 or 400 is mounted on a patient.

The position and angle of trajectory of guide member 60, 260, 360, 460 coupled to first frame member 20, 220, 320, 420 may then be set based on its relationship to a pre-placed burr hole. Preferably, guide member 60, 260, 360, 460 is aligned with a burr hole previously drilled into the patient's cranium. For placement of an external ventricular drain, the burr hole may be placed at Kocher's point anterior to the coronal suture, which measured from the nasion cephalad, is about 10 cm to about 11 cm along the midline and about 3 cm to the patient's right. To align guide member 60, 260, 360, 460 relative to a pre-drilled burr hole, first frame member 20, 220, 320, 420 is positioned in the posterior to anterior direction so as to be aligned with the burr hole. Preferably, first frame member 20, 220, 320, 420 is positioned about 40° to about 65°, preferably, about 40° to about 55° or about 55° to about 65° from the nasion. With respect to surgical guides 200, 300, first frame member 220, 320 may be pivotally and adjustably positioned relative to second frame member 280, 380 to align first frame member 220, 320 relative to the burr hole. The surgeon may precisely select and lock the angle between second frame member 280, 380 and first frame member 220, 320 using adjustment mechanism 294, 394. In an exemplary embodiment, first frame member 220, 320 is preferably positioned about 55° to about 65° with respect to the nasion.

Guide member 60, 260, 360, 460 is then slidably positioned along first frame member 60, 220, 320, 460 in a medial to lateral dimension so as to be aligned with the burr hole. Thus positioned, guide member 60, 260, 360, 460 establishes both the medial-lateral and posterior-anterior angular directions necessary to reach the ventricle. In an exemplary embodiment, guide member 60, 260, 360, 460 specifically passage 62, 262, 362, 462 of guide member 60, 260, 360, 460 may be positioned about 3.5 cm to about 7 cm from the midsagittal plane.

When using surgical guides 200, 300, the location of first frame member 220, 320 and guide member 260, 360 may be adjusted based on CT data of a patient's head to more precisely position guide member 260, 360 to ensure a successful placement of surgical instrument 210, 310.

By virtue of orienting the longitudinal axis of passage 62, 262, 362, 462 or at least a longitudinal axis of a distal section 70, 270, 370, 470 of passage 62, 262, 362, 462 within a plug plane and by virtue of placing plugs 40, 42, 240, 242, 340, 342, 440, 442 within a patient's external auditory meatuses, the longitudinal axis of passage 62, 262, 362, 462 or at least a longitudinal axis of a distal section 70, 270, 370, 470 of passage 62, 262, 362, 462 is also oriented within an EAM plane. This orientation of guide member 60, 260, 360, 460 establishes an appropriate trajectory at which surgical instrument 10, 210, 310, 410 may be inserted through the burr hole in order to reach an intracranial ventricle. At least a portion of surgical instrument 10, 210, 310, 410 may then be placed within the intracranial ventricle.

For the placement of an external ventricular drain, a needle is inserted within a proximal opening 65, 265, 365, 465 of guide member passage 62, 262, 362, 462. The needle, directed by the orientation of passage 62, 262, 362, 462, namely the distal section 70, 270, 370, 470 of passage 62, 262, 362, 462, is inserted through the burr hole at a trajectory that orients a distal portion of guide member passage 62, 262, 362, 462 in both a plug plane and an EAM plane. A distal tip of the needle is then placed into the burr hole about 3 cm in depth. When the needle enters the foramen of monro of the lateral ventricle, cerebral spinal fluid is refluxed, indicating the appropriate placement of the needle within the intracranial ventricle. A catheter may then be inserted through passage 62, 262, 362, 462 over the needle, which functions as an additional insertion guide. Alternatively, without previously inserting a needle, a catheter may be directly inserted into passage 62, 262, 362, 462 and through the burr hole to a depth of about 3 cm. By virtue of the pre-set angle established by the surgical guide, the catheter and/or needle is thus guided to the expected location of the ventricle.

If no cerebral spinal fluid is refluxed when a needle or catheter is first inserted, it may be concluded that the location of the intracranial ventricle has been distorted by intracranial masses, such as a hematoma. In such cases, a CT scan of a patient's head may be used to locate the abnormally situated intracranial ventricle. Surgical guide 200 of the present invention may then be used as a coordinate system to more accurately guide surgical instrument 210 in a medial to lateral direction and in a posterior to anterior direction for the precise location of the intracranial ventricle.

Once the catheter is appropriately positioned within the intracranial ventricle, surgical guide 100, 200, 300 or 400 may be detached from the drainage catheter without affecting the position or requiring movement of the catheter. Preferably, the catheter remains in place while the surgical guide 100, 200, 300 or 400 is withdrawn from around the catheter, such that catheter passes through a side opening 69, 269, 369, 469 of surgical guide 100, 200, 300 or 400. The catheter is thus positionally fixed and attached to a dedicated drainage tubing, and the separated surgical guide may be subsequently discarded.

In an exemplary embodiment, in addition to functioning as a placement guide for surgical instrument 10, certain embodiments of the present invention, such as surgical guides 200, 300 and 400, may also be used as a guide for drilling the burr hole. An appropriate burr hole drill site may be established based on the identified location of the intracranial ventricle. Preferably, the drilling location may be identified using the present invention by triangulating: the distance from the intracranial ventricle to a midsagittal plane using reference indicators 232, 332, 432 the distance from the intracranial ventricle to the nasion using second frame member 280, 380, 480 and the distance from the intracranial ventricle to the external auditory meatuses using plugs 240, 242, 340, 342, 440, 442. For the placement of an external ventricular drain, the burr hole is typically placed at a location about 10 cm to about 12.5 cm cephalad from the nasion (or about 1 cm or more anterior to the coronal suture) and about 3 cm to about 4 cm to the right of the midpupillary or midsagittal plane. When used as a drill guide, guide member 260, 360, 460 may be configured to receive and removably secure a drill and/or drill bit to form a burr hole. Alternatively, a special guide member 260, 360, 460 adapted to receive a drill and/or drill bit may be removably attached to first frame member 220, 320, 420. In an exemplary embodiment, a surgeon may be able to use an adjustable surgical guide, such as surgical guide 200, 300, as a coordinate system to locate an appropriate site to burr hole drill site based on a CT scan of a patient's cranium and a 3D image processing system that precisely identifies and provides the coordinates locating the intracranial ventricle.

The method of the present invention may be used for a wide variety of applications, such as the accurate placement of an external ventricular drain, including drain placement in cases where the ventricles are shifted due to intracranial masses. While the invention is well suited to enabling the bedside placement of an external ventricular drain or the ventricular catheter of a ventriculoperitoneal shunt under emergency situations, it can also be used with little modification for placement of a burr hole for a subdural evacuation device or small craniotomy in patients with extra-axial hemorrhage or intra-axial masses in circumstances where frameless or frame stereotaxy is not available. The surgical guide of the present invention may also be used for other surgical procedures and may enable intracranial placement of a wide variety of surgical instruments. For example, the invention may be used to facilitate placement of a chemotherapy catheter with a reservoir.

The features of the invention are further illustrated in the following non-limiting example.

### EXAMPLE

In a preliminary study, the accuracy with which the surgical guide of the present invention is able to place a needle within a lateral ventricle was investigated. A surgical guide including a U-shaped first frame member, two external auditory meatuses plugs and a guide member slidably coupled to the first frame member, as shown in FIG. 1, was mounted on a cadaver skull filled with a stiff foam intended to represent the brain. The plugs were securely inserted within the external auditory meatuses. Upon aligning the guide member with a burr hole located about 10 cm from the nasion and about 3 cm from the midsagittal plane, a 22 gauge needle was then inserted through a linear passage of the guide member and through the burr hole. The location of the burr hole was established based on a pre-determined angle obtained from population studies. The location of the needle tip was then recorded. The same procedure was subsequently repeated 4 times.

The variation in needle tip position in the skull in the anterior to posterior dimension was within a circle of about 6 mm in diameter. Specifically, the displacement of the needle tip in the anterior to posterior dimension for each of the 5 passes was about 64 mm, 58 mm, 61 mm, 67 mm and 56 mm The lateral displacement from the midline was within a circle of about 9 mm diameter located at the midline. The lateral displacement from the midline was about 0 mm, 7 mm, 8 mm, 10 mm, and −8 mm It is believed that the stiff foam material used to fill the skull, which was estimated to be firmer than brain matter, and the relatively gauge of the needle caused the needle to self-steer away from the needle bevel, accounting for half of the displacements.

Even under the less than ideal conditions of the study, the results indicate that the surgical guide was able to successfully place the needle within the intended target site, the foramen of monro of the lateral ventricle.

It is to be understood that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A surgical guide comprising:
a first frame member having a substantially U-shaped configuration adapted to be positioned over a portion of an individual's head;
two plugs attached to the first frame member, each plug including a portion adapted to be positioned within an individual's external auditory meatuses; and
a guide member positioned along a portion of the first frame member, wherein the guide member includes a passage for receiving a surgical instrument and wherein the first frame member and the guide member cooperate to ensure that a longitudinal axis of a distal portion of the passage is oriented in a plug plane that passes through a line intersecting a portion of each the two plugs when positioned within the external auditory meatus.

2. The surgical guide of claim 1, wherein the longitudinal axis of the entire passage is oriented within the plug plane.

3. The surgical guide of claim 1, wherein a length of the passage is partially formed in and penetrates an exterior surface of the guide member.

4. The surgical guide of claim 3, wherein the guide member comprises a clamp for releasably securing the surgical instrument within the passage.

5. The surgical guide of claim 3, wherein the guide member is slidably coupled to the first frame member and comprises a securing member for releasably securing the guide member to a location along the first frame member.

6. The surgical guide of claim 1, wherein the first frame member is rigidly connected to the two plugs.

7. The surgical guide of clam 1, further comprising a second frame member having a substantially U-shaped configuration which is pivotally coupled to the first frame member, wherein the second frame member is adapted to be positioned against an individual's nasion.

8. The surgical guide of claim 7, further comprising structure to limit pivotal adjustment of a position of the first member relative to the second frame member such that an angle formed between the first and second frame members is about 40° to about 65°.

9. The surgical guide of claim 8, further comprising a ratchet for pivotally adjusting the position of the first frame member relative to the second frame member, wherein the ratchet comprises a plurality of fasteners that may be removably fastened to a mating structure positioned on the first or second frame member.

10. The surgical guide of claim 1, further comprising a second frame member having a U-shaped configuration, wherein the first frame member and second frame member are fixed relative to one another such that an angle formed between the first and second frame members is about 40° to about 65°.

11. A method for placing a portion of a surgical instrument in an intracranial ventricle of an individual comprising the steps of:
a) positioning the two plugs of the surgical guide as claimed in claim 1 within the individual's external auditory meatuses whereby at least the distal portion of the passage in the guide member is located in a plane passing through a longitudinal axis of the individual's external auditory meatuses allowing for insertion of the surgical instrument at a desired trajectory;
b) aligning the guide member with a burr hole in an individual's cranium;
c) inserting the surgical instrument through the passage and into the burr hole; and
d) positioning the portion of the surgical instrument within the intracranial ventricle.

12. The method of claim 11, wherein the surgical instrument is selected from the group consisting of: a catheter, a shunt and a needle.

13. The method of claim 11, further comprising the step of aligning the first frame member at an angle of about 40° to about 65° relative to a nasion of the individual.

14. The method of claim 11, wherein the surgical guide further comprises a second frame member and wherein the method further comprises placing the second frame member of the surgical guide against an individual's nasion.

15. The method of claim 14, wherein the first frame member and second frame member are fixed relative to one another such that an angle formed between the first and second frame members is about 40° to about 65°.

16. The method of claim 14, further comprising a step of pivotally adjusting the position of the first frame member relative to the second frame member such that an angle formed between the first and second frame members is about 40° to about 65°.

17. The method of claim 14, further comprising a step of identifying a location of the intracranial ventricle by imaging an individual's head and wherein step b further comprises adjusting the location of the guide member based on an image of the individual's head so that the guide member is aligned with the intracranial ventricle.

18. The method of claim 11, further comprising the step of detaching the surgical guide from the individual without removing the surgical instrument positioned in the burr hole.

19. A method for accessing an intracranial ventricle of an individual comprising the steps of:
a) positioning the two plugs of the surgical guide as claimed in claim 1 within the individual's external auditory meatuses, whereby at least the distal portion of the passage in the guide member is located in a plane passing through a longitudinal axis of the individual's external auditory meatuses to allow for insertion of the surgical instrument at a desired trajectory;
b) identifying a location of the intracranial ventricle;
c) identifying a burr hole drill site based on the identified location of the intracranial ventricle;
d) aligning the guide member with the burr hole drill site; and
e) inserting a drill bit into the passage and drilling a burr hole in an individual's cranium.

20. The method of claim 19, wherein step b further comprises identifying the location of the intracranial ventricle by imaging an individual's head and wherein step c further comprises using the surgical guide to map the location of the burr hole drill site.
